(19) United States
(12) Patent Application Publication
(10) Pub. No.: US 2022/0023348 A1
(43) Pub. Date: Jan. 27, 2022

(54) GENETICALLY MODIFIED HSPCS RESISTANT TO ABLATION REGIME

(71) Applicant: Forty Seven, Inc., Foster City, CA (US)

(72) Inventors: Craig Gibbs, Palo Alto, CA (US); Jens-Peter Volkmer, Menlo Park, CA (US); Irving L. Weissman, Stanford, CA (US)

(21) Appl. No.: 17/291,397

(22) PCT Filed: Nov. 26, 2019
(86) PCT No.: PCT/US19/63402

§ 371 Date: May 5, 2021

Related U.S. Application Data
(60) Provisional application No. 62/772,545, filed on Nov. 28, 2018.

(51) Int. Cl. A61K 35/28; C12N 5/0789; C07K 16/28

(52) U.S. Cl. CPC A61K 35/28; C12N 2510/00; C07K 16/2803; C12N 5/0647

(57) ABSTRACT

The invention provides genetically modified hematopoietic stem or progenitor cells (HSPCs) and methods of using the HSPCs in stem cell replacement therapy. The HSPCs are genetically modified to express a receptor conferring a selective advantage on the introduced cells relative to endogenous HSPCs or a control HSPCs without the modification. The presence of such a receptor provides resistance to an immunotherapy regime used for eliminating endogenous HSPCs.

### CROSS-REFERENCE TO RELATED APPLICATION

The present application claims the benefit of U.S. 62/772,545, filed Nov. 28, 2018, which is incorporated by reference in its entirety for all purposes.

### REFERENCE TO A SEQUENCE LISTING

The application includes sequences disclosed in txt file 53950-SEQLST, of 11 kbytes, created Nov. 26, 2019, which is incorporated by reference.

### BACKGROUND

Stem cells provide the means for organisms to maintain and repair certain tissues, through propagation to generate differentiated cells. Hematopoietic stem cell transplantation has been used to provide patients with the capacity to generate blood cells, usually where the patient has been ablated of endogenous hematopoietic stem cells by chemotherapy, or other conditioning regime.

Hematopoietic cell transplantation generally involves the intravenous infusion of autologous or allogeneic blood forming cells including hematopoietic stem cells. These are collected from bone marrow, peripheral blood, or umbilical cord blood and transplanted to reestablish hematopoietic function in patients whose bone marrow or immune system is damaged or defective. This procedure is often performed as part of therapy to eliminate a bone marrow infiltrative process, such as leukemia, or to correct congenital immunodeficiency disorders. Hematopoietic cell transplantation is also used to allow patients with cancer to receive higher doses of chemotherapy than bone marrow can usually tolerate; bone marrow function is then salvaged by replacing the marrow with previously harvested stem cells (see generally WO 2004/002425 and WO2018/140940).

### SUMMARY OF THE CLAIMED INVENTION

The invention provides a hematopoietic stem or progenitor cell (HSPC) genetically modified to express a receptor conferring a selective proliferation advantage on the genetically modified HSPC on introduction into a subject relative to endogenous HSPCs. Optionally, the cell is a primitive stem cell.

The invention further provides a population of at least 10⁵ HSPCs as defined above or below. Optionally, the at least 10⁵ HSPCs are CD34⁺. Optionally, the population is clonal. Optionally, the population includes primitive stem cells and common progenitor cells.

In some cells, the receptor is any of B2M/MHC-1, PD-L1, CD24, GAS6, CD47, c-Kit or a combination of multiple such receptors. In some cells, the receptor is a mutant form of the receptor, wherein the mutant has reduced binding to an antibody relative to the wildtype form of the receptor. In some cells, the receptor is CD47 or c-Kit. Some cells are further genetically modified to express a functional human protein as a result of which the HSPC can alleviate a genetic disorder. The genetic disorder can be due to mutation of a gene encoding the human protein in subjects having the disorder. In some cells, the human protein is a hemoglobin.

Some HSPCs are genetically modified by homologous recombination between a targeting construct and am endogenous locus. In some HSPCs the genetic modification is heterozygous. In some HSPCs, the genetic modification is homozygous.

The invention further provides a method of modifying an HSPC comprising introducing into the HSPC a construct that is incorporated into the genome of the HSPC forming a transcriptional unit that can express a receptor conferring a selective proliferation advantage on the genetically modified HSPC relative to the HSPC before modification. Optionally, the construct comprises a transcriptional unit comprising a segment encoding the receptor operably linked to regulatory sequences for its expression. Optionally, the construct undergoes homologous recombination with an endogenous locus. Optionally, the method comprises introducing a nuclease into the HSPC, which cleaves genomic DNA proximate to the locus of the homologous recombination thereby stimulating the homologous recombination. Optionally, the nuclease is introduced by introducing a construct encoding the nuclease, which is expressed in the HSPC.

The invention further provides a method of treating a subject, comprising (a) administering an immunotherapeutic agent specifically binding to c-Kit to deplete endogenous HSPCs expressing c-Kit; and (b) administering replacement HSPCs genetically modified to express a receptor conferring a selective proliferation advantage on the genetically modified HSPCs relative to endogenous HSPCs and thereby resist depletion by the immunotherapeutic agent specifically binding to c-Kit, wherein the replacement HSPCs at least partially replace the endogenous HSPCs. Optionally, the receptor conferring a selective proliferation advantage is CD47. Optionally, the CD47 receptor contains a mutation and the method further comprises administering an antibody or SIRPα Fc fusion protein that binds to wildtype CD47 and antagonizes its interaction with SIRPα more strongly over its binding and antagonism, if any, of the mutated receptor to SIRPα. Optionally, endogenous HSPCs are only partially depleted before performing step (b). Optionally, step (a) is performed before step (b). Optionally, step (a) is performed at the same time or after step (b). Optionally, the immunotherapeutic agent specifically binding to c-Kit is detectable in the serum when the introducing step is performed. Optionally, the immunotherapeutic agent specifically binding to c-Kit is administered on multiple occasions before and after step (b). Optionally, the immunotherapeutic agent specifically binding to c-Kit is an antibody. Optionally, the antibody has an Fc domain effective to promote ADCC or ADP.

Optionally, the subject has a genetic disorder of a type of blood cell and the replacement HSPCs develop into blood cells of the type free of the disorder. Optionally, the replacement HSPCs are autologous cells which have been further genetically modified to be free of the disorder. Optionally, the genetic disorder is sickle cell anemia. Optionally, the subject has a cancer. Optionally, the cancer is of a blood cell, which expresses c-Kit or derives from a HSPC expressing c-Kit. Optionally, the subject has a cancer and has received chemotherapy against the cancer. Optionally, the subject receives an organ transplant after step (a).

### DEFINITIONS

A subject includes both humans being treated by the disclosed methods and other animals, particularly mammals, including pet and laboratory animals, e.g. mice, rats, rabbits. Thus the methods are applicable to both human therapy and veterinary applications.

An immunotherapeutic agent refers to an antibody or Fc-fusion protein against a designated target. For example antibodies against CD47 and a SIRPα-Fc fusion are immunotherapeutic agents against CD47.

Operable linkage of nucleic acid or amino acid sequences means that the sequences are linked such that each can perform its intended function. For example, operable linkage of a promoter to a coding sequence implies the coding sequence can be expressed from the promoter. Operable linkage of a protein to a signal peptide implies the signal peptide can direct secretion of the protein or target it for incorporation into a cell membrane.

A functional nucleic acid or protein encoded by the nucleic acid refers to a wildtype form of the nucleic acid, or natural or induced variant thereof that can support normal physiology of a subject expressing the nucleic acid to the protein. In other words, expression of the nucleic acid in subjects does not result in a pathological condition with partial or complete penetrance. For example, a variant of a wildtype nucleic acid or protein including one or more variations not having any significant effect on wildtype function would be considered a functional nucleic acid or protein. Such is to be contrasted with a nucleic acid including a mutation or protein expressed therefrom, whose presence in a subject in heterozygous or homozygous form is associated with development of a pathological condition with partial or complete penetrance. A functional nucleic acid introduced into replacement HSPCs such that can express its encoded protein can thus at least partly alleviate pathology resulting from a mutated form of the nucleic acid and/or protein in endogenous HSPCs.

Immunotherapeutic agents are typically provided in isolated form. This means that such an agent is typically at least 50% w/w pure of interfering proteins and other contaminants arising from its production or purification but does not exclude the possibility that the agent is combined with an excess of pharmaceutical acceptable carrier(s) or other vehicle intended to facilitate its use. Sometimes agents are at least 60, 70, 80, 90, 95 or 99% w/w pure of interfering proteins and contaminants from production or purification. Often an agent is the predominant macromolecular species remaining after its purification.

Specific binding of immunotherapeutic agent to its target antigens means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. An immunotherapeutic agent specifically binding to its target antigen can also be described as being against its target antigen.

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact signal sequences are cleaved once antibodies or other immunotherapeutic agents of the invention have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. In IgA, the heavy constant region is divided into CH1, CH2 and CH3. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcRn binding.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites, i.e., is divalent. In natural antibodies, the binding sites are the same. However, in bispecific the binding sites are different (see, e.g., Songsivilai and Lachmann, Clin. Exp. Immunol., 79:315-321 (1990); Kostelny et al., J. Immunol., 148:1547-53 (1992)). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989). Kabat also provides a widely used numbering convention (Kabat numbering) in which corresponding residues between different heavy chain variable regions or between different light chain variable regions are assigned the same number. Although Kabat numbering can be used for antibody constant regions, the EU index is more commonly used, as is the case in this application.

The term “epitope” refers to a site on an antigen to which an arm of a bispecific antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. Some antibodies bind to an end-specific epitope, meaning an antibody binds preferentially to a polypeptide with a free end relative to the same polypeptide fused to another polypeptide resulting in loss of the free end. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

The term “antigen” or “target antigen” indicates a target molecule bound by one binding site of a bispecific antibody. An antigen may be a protein of any length (natural, synthetic or recombinantly expressed), a nucleic acid or carbohydrate among other molecules. Antigens include receptors, ligands, counter receptors, and coat proteins.

Antibodies that recognize the same or overlapping epitopes can be identified in a simple immunoassay showing the ability of one antibody to compete with the binding of another antibody to a target antigen. The epitope of an antibody can also be defined X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2.times., 5.times., 10.times., 20.times. or 100.times.) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention for a variable region or EU numbering for a constant region. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

The term “antibody-dependent cellular cytotoxicity”, or ADCC, is a mechanism for inducing cell death that depends upon the interaction of antibody-coated target cells (i.e., cells with bound antibody) with immune cells possessing lytic activity (also referred to as effector cells). Such effector cells include natural killer cells, monocytes/macrophages and neutrophils. ADCC is triggered by interactions between the Fc region of an antibody bound to a cell and Fcγ receptors, particularly FcγRI and FcγRIII, on immune effector cells such as neutrophils, macrophages and natural killer cells. The target cell is eliminated by phagocytosis or lysis, depending on the type of mediating effector cell. Death of the antibody-coated target cell occurs as a result of effector cell activity.

The term “antibody-dependent cellular phagocytosis,” or ADCP, refers to the process by which antibody-coated cells are internalized, either in whole or in part, by phagocytic immune cells (e.g., macrophages, neutrophils and dendritic cells) that bind to an immunoglobulin Fc region.

The term “complement-dependent cytotoxicity” or CDC refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target cells bind and activate complement component C1q which in turn activates the complement cascade leading to target cell death. Activation of complement may also result in deposition of complement components on the target cell surface that facilitate ADCC by binding complement receptors (e.g., CR3) on leukocytes.

### DETAILED DESCRIPTION

I. General

The invention provides genetically modified hematopoietic stem or progenitor cells (HSPCs) and methods of using the HSPCs in stem cell replacement therapy. The HSPCs are genetically modified to express a receptor conferring a selective advantage on the introduced cells relative to endogenous HSPCs or a control HSPCs without the modification. The presence of such a receptor provides resistance to an immunotherapy regime used for eliminating endogenous HSPCs. Thus, the immunotherapy regime favors propagation of introduced HSPCs relative to endogenous HSPCs. The genetically modified HSPCs can be used to replace endogenous HSPCs subject to a genetic disorder, to replace endogenous HSPCs subject to a hematologic malignancy or autoimmune disease, to replace endogenous HSPCs damaged by a chemotherapy regime, or to replace endogenous HSPCs ablated prior to organ transplant, among other applications.

II. HSPCs

Depending on the application, the HSPCs to be introduced into a subject can be autologous (i.e., from that subject), allogenic (from another individual of the same species), or xenogenic (from a different species). If allogenic, the HSPCs can be matched fully or partially or unmatched for MHC alleles. Matched HSPCs can be obtained from a relative or a stranger.

Although all HSPCs are capable of propagation and differentiation into cells of myeloid or lymphoid linages or both, HSPCs include cells at different stages of differentiation. Primitive stem cells can propagate indefinitely and form all cells types of myeloid and lymphoid lineages. Primitive stem cell differentiate into multi-potent progenitors, which can give rise to all cells of both myeloid and lymphoid lineages but cannot propagate indefinitely. Multipotent progenitors give rise to oligo-potent progenitors including the common lymphoid progenitor, CLP, which gives rise to mature B lymphocytes, T lymphocytes, and natural killer (NK) cells. Multipotent progenitors also give rise to the common myeloid progenitor (CMP) which further differentiates into granulocyte-macrophage progenitors, which differentiate into monocytes/macrophages and granulocytes, and megakaryocyte/erythrocyte progenitors, which differentiate into megakaryocytes/platelets and erythrocytes (see FIG. 1 of Bryder et al., Am. J. Pathol. 169, 338-346 (2006)).

Primitive HC and multipotent progenitor cells can be distinguished from each other experimentally, for example, by performing a Cobblestone-Forming Area Cell Assay (Ploemacher et al. Blood. 78:2527-33 (1991)). Progenitor cells appear earlier, over a 1 to 3 week period in culture whereas the primitive hematopoietic stem cells appear at 4 to 5 weeks in culture. Both primitive stem cells and multipotent progenitor cells are useful for replacement therapy. Further differentiated cells such as the CMP or CLP can also be used but may be less versatile because of their limited propagation ability and restricted lineage of cells they are capable of forming.

HSPCs can be obtained by harvesting from bone marrow, from peripheral blood or umbilical cord blood. Bone marrow is generally aspirated from the posterior iliac crests while the donor is under either regional or general anesthesia. Additional bone marrow can be obtained from the anterior iliac crest. Bone marrow can be primed with granulocyte colony-stimulating factor (G-CSF; filgrastim [Neupogen]) to increase the stem cell count. Reference to “whole bone marrow” generally refers to a composition of mononuclear cells derived from bone marrow that have not been selected for specific immune cell subsets. “Fractionated bone marrow” may be, for example, depleted of T cells, e.g. CD8+ cells, CD52+ cells, CD3+ cells, etc.; enriched for CD34+ cells, and so forth.

HSPCs can also be obtained by mobilization of stem cells from the bone marrow into peripheral blood by cytokines such as G-CSF, GM-CSF or Plerixafor (also known as AMD3100 or Mozobil). An exemplary dose of G-CSF used for mobilization is 10 μg/kg/day but higher doses can be given up to e.g., 40 μg/kg/day can be given. Mozobil may be used in conjunction with G-CSF to mobilize HSPC to peripheral blood for collection. HSPCs can be harvested from peripheral blood with an apheresis device.

HSPCs can also be obtained from umbilical cord blood (UBC) typically for allogenic transplant. UCB is enriched in primitive stem/progenitor cells able to produce in vivo long-term repopulating stem cells.

Blood cells isolated from these procedures can undergo enrichment for HSPCs or a subset thereof, e.g., primitive stem cells and/or common progenitor by affinity enrichment for characteristic cell surface markers. Such markers include CD34; CD90 (thy-1); CD59; CD110 (c-mpl); c-Kit (CD-117). Cells can be selected by affinity methods, including magnetic bead selection, flow cytometry, and the like from the donor hematopoietic cell sample. Several immunoselection devices, including Ceparte, Isolex 300i, and CliniMACS Prodigy® are commercially available for CD34+ cell selection.

The HSPC composition can be at least about 50% pure, as defined by the percentage of cells that are CD34+ in the population, may be at least about 75% pure, at least about 85% pure, at least about 95% pure, or more (likewise defined).

III. Receptors Conferring Resistance to an Ablative Regime

HSPCs are genetically engineered to express one or more receptors (conferring a selective proliferation advantage on replacement HSPCs over endogenous HSPCs such that after introduction of replacement HSPCs into a subject containing endogenous HSPCs, the proportion of replacement HSPCs will increase over time. Suitable receptors include don't eat me receptors. A don't eat me receptor is a receptor that protects a cell expressing the receptor from the immune system of the organism in which the cell typically resides. In the present methods, a don't eat me receptor protects replacement HSPCs against immune response in a recipient subject, particularly against immunotherapy used in ablating endogenous HSPCs from the subject. Examples of suitable receptors are CD47 (e.g., Swiss Prot Q08722), c-Kit (e.g., Swiss Prot P10721), β2M (e.g., Swiss Prot P61769)/MHC-1 (many different accession numbers), PD-L1 (Q91\12Q7), CD24 (Swiss Prot P25063), GAS6 (e.g., Swiss Prot Q14393) and CD31 (e.g., Swiss Prot P16284). Reference to such receptors should be understood as referring to the human forms, such as those of the accession numbers provided. However, non-human forms of these receptors can also be used in veterinary applications or modelling experiments.

Binding of CD47 on HSPCs to SIRPα on phagocytic cells generates a don't eat me signal protecting the HSPCs from phagocytosis including that induced by antibodies used in an ablation regime for endogenous HSPCs.

CD31 is another example of a don't eat me receptor. Brown et al., Nature. 2002; 418(6894):200-203.

MHC class I molecules are heterodimers that consist of two polypeptide chains, α and β2-microglobulin (b2m). The two chains are linked noncovalently via interaction of b2m and the α3 domain. MHC class I proteins generate a “don't eat me” signal by binding to a protein called LILRB1 on macrophages. When either the MHC class I proteins or LILRB1 is blocked, the “don't eat me” signal was lifted and the macrophages' ability to kill cells bearing the MHC class I was restored. MHC class I can also serve as an inhibitory ligand for natural killer cells (NKs). Reduction in the normal levels of surface class I MHC, a mechanism employed by some viruses and certain tumors to evade CTL responses, activates NK cell killing.

Programmed death-ligand 1 (PD-L1) provides a “don't find me” signal to the adaptive immune system.

Growth arrest-specific 6, also known as GAS6, is a human gene coding for the Gas6 protein. GAS6 is expressed on certain cancers including AML. WU et al., Cell Death & Disease volume 8, page e2700 (2017)).

CD24 is a small cell surface protein expressed by various cancers and cancer stem cells and is involved in cell adhesion and cancer metastasis. Jaggupilli et al., Clinical and Developmental Immunology Volume 2012, Article ID 708036.

Protective receptors as described above can be in the form of wildtype sequence (typically human) or mutant versions of such sequences. If not wildtype, receptors typically show at least 90, 95 or 99% sequence identity to the wildtype over the full length of the variant or wildtype, whichever is shorter). Any differences can but need not be conservative substitutions. Protective receptors, whether of wildtype or mutant sequence, typically include extracellular, transmembrane and intracellular domains. Protective receptors can be full length (with the possible exception of a signal peptide) or can be truncated (e.g., from the ends) provided a protective function is retained. For example, if the protective role involves ligand binding such as between CD47 and SIRPα, such binding should be retained.

Wildtype versions of protective receptors are particularly useful when the ablation regime does not include an antibody against the protective receptor. For example, if the ablative regime uses an antibody against c-Kit but not against CD47, then wildtype CD47 can be used as a protective receptor on replacement HSPCs.

Mutant versions of protective receptors are particularly useful for receptors against which antibodies are directed either as part of an ablation regime against endogenous HPSCs or against cancer cells bearing the receptor. The introduction of the mutation into the protective receptor reduces or eliminates binding of the receptor to an antibody or other immunotherapeutic agent against the receptor used for ablation of endogenous HSPCs or cancer treatment. Likewise, the mutation reduces the ability of the antibody or other immunotherapeutic agent to antagonize the receptor. In other words, the antibody or other immunotherapeutic agent binds to and antagonizes more strongly the wildtype receptor over the mutant receptor (if at all). The mutation can be present in one or more amino acid positions of the protective receptor forming the epitope bound by such an antibody. For example, if the ablation regime involves an antibody against c-Kit binding to an epitope X, then HSPCs can be genetically manipulated to express c-Kit with a mutation in epitope X such that the antibody does not bind or binds only to a reduced extent to the mutated c-Kit. Alternatively, the mutation can reduce or eliminate antibody binding allosterically. Such a mutation preferably does not significantly reduce binding of c-Kit to its ligand stem cell factor. Likewise, if the ablation regime involves an antibody against CD47 binding to epitope Y to promote ablation of endogenous HSPC, then HSPCs can be genetically modified to express CD47 mutated within epitope Y, such that the antibody does not bind or binds at only a reduced extent to CD47. Alternatively, the mutation can reduce or eliminate an anti-CD47 antibody binding allosterically. Such a mutation preferably does not significantly reduce binding of CD47 to SIRPα. Analogously mutated forms of other protective receptors can likewise be used in combination with an ablation or anti-cancer therapy involving an antibody against one or more of such receptors.

IV. Ablation Regimes

Ablation regimes serve to reduce or eliminate endogenous HSPCs. Endogenous HSPCs can be reduced by a factor of e.g., at least 10%, 25%, 50% or 90% before introducing replacement HSPCs. Some regimes do not reduce endogenous HSPCs by more than, e.g., 50%, 25% or 10% before introducing replacement HSPCs.

Such ablation regimes involve administration of an antibody specifically binding to c-Kit (CD117) (see generally WO 2008067115). C-Kit is a cell surface marker used to identify certain types of HSPCs in the bone marrow. Hematopoietic stem cells (HSC), multipotent progenitors (MPP), and common myeloid progenitors (CMP) express high levels of c-Kit. Such antibodies can reduce endogenous HSPCs by inhibiting interaction between c-Kit and its ligand and by effector mediated mechanisms, such as ADCC, ADCP and CDC. c-Kit is a receptor tyrosine kinase type III, which binds to stem cell factor (a substance that causes certain types of cells to grow), also known as “steel factor” or “c-Kit ligand.” When this receptor binds to stem cell factor, it forms a dimer that activates its intrinsic tyrosine kinase activity, which in turn phosphorylates and activates signal transduction molecules that propagate the signal in the cell. A number of antibodies that specifically bind human c-Kit are commercially available, including SR1, 2B8, ACK2, YB5-B8, 57A5, 104D2 (US20180214525). AMG191 is a humanized form of SR1 (U.S. Pat. Nos. 8,436, 150, and 7,915,391). Further humanized forms of SR1 are described in PCT/US19/63091, filed Nov. 25, 2019 incorporated by reference in its entirety for all purposes. Some antibodies of the invention have a mature heavy chain variable region having a sequence of any of the chains designated SEQ ID NOS. 13, 17 or 21 designated AH2, AH3, and AH4 respectively of PCT/US19/63091 filed Nov. 25, 2019 and a mature light chain variable region having a sequence of SEQ ID NO: 53, NL2, of PCT US2019/US19/63091 (SEQ ID NOS: 13-16 herein). Any of these antibodies, including chimeric, veneered or humanized forms, or antibodies binding the same epitope or competing therewith for binding to c-Kit can be used in the disclosed methods. Other antibodies against c-Kit can be generated de novo by standard immunological techniques as further described below.

The ablation regime can also include an immunotherapeutic agent inhibiting CD47-SIRPα interaction for use in combination with an antibody against c-Kit (see generally WO2016033201). Such an agent promotes effector mediated elimination of endogenous HSPCs mediated by anti-c-Kit. Such agents include antibodies specifically binding to CD47 or SIRPα. Such agents also include a CD47 ECD fused to an Fc, which functions similarly to antibodies against SIRPα, or a SIRPα fused to an Fc, which functions similarly to antibodies against CD47. (see Zhang et al., Antibody Therapeutics, Volume 1, Issue 2, 21 Sep. 2018, Pages 27-32). Preferred antibodies antagonize CD47-SIRPα interaction without conferring an activating signal through either receptor.

Examples of suitable anti-CD47 antibodies include clones B6H12, 5F9, 8B6, C3, (for example as described in WO 2011/143624) CC9002 (Vonderheide, Nat Med 2015; 21: 1122-3., 2015), and SRF23 (Surface Oncology). Suitable anti-CD47 antibodies include human, humanized or chimeric versions of such antibodies, antibodies binding to the same epitope or competing therewith for binding to CD47. Humanized antibodies (e.g., hu5F9-IgG4-WO2011/143624) are especially useful for in vivo applications in humans due to their low antigenicity. Similarly caninized, felinized antibodies and the like are especially useful for applications in dogs, cats, and other species respectively. Some humanized antibodies specifically binds to human CD47 comprising a variable heavy (VH) region containing the VH complementarity regions, CDR1, CDR2 and CDR3, respectively set forth in SEQ ID NO: 20, 21 and 22; and a variable light (VL) region containing the VL complementarity regions, CDR1, CDR2 and CDR3, respectively set forth in SEQ ID NO:23, 24 and 25 of WO2011/143624 (SEQ ID NOS:1-6 herein). Some humanized antibodies include a heavy chain variable region selected from SEQ ID NO: 36, SEQ ID NO: 37 and SEQ ID NO: 38 and a light chain variable region selected from SEQ ID NO: 41, SEQ ID NO: 42 and SEQ ID NO: 43 set forth in W02011/143624 (SEQ ID NOS. 7-12 herein). An exemplary antibody is magrolimab.

Suitable anti-SIRPα antibody specifically bind SIRPα, preferably without activating/stimulating enough of a signaling response to inhibit phagocytosis, and inhibit an interaction between SIRPα and CD47. Suitable anti-SIRPα antibodies include fully human, humanized or chimeric versions of such antibodies. Exemplary antibodies are KWAR23 (Ring et al., Proc Natl Acad Sci USA 2017 Dec. 5; 114(49): E10578-E10585, WO2015/138600), MY-1 (Yanagita et al., JCI Insight. 2017 Jan. 12; 2(1): e89140), and Effi-DEM (Zhang et al., Antibody Therapeutics, Volume 1, Issue 2, 21 Sep. 2018, pages 27-32). Humanized antibodies are especially useful for in vivo applications in humans due to their low antigenicity. Similarly caninized, felinized, and the like antibodies are especially useful for applications in dogs, cats, and other species respectively.

Immunotherapeutic agents also include soluble CD47 polypeptides that specifically binds SIRPα and reduce the interaction between CD47 on an HSPC and SIRPα on a phagocytic cell (see, e.g., WO2016179399). Such polypeptides can include the entire ECD or a portion thereof with the above functionality. A suitable soluble CD47 polypeptide specifically binds SIRPα without activating or stimulating signaling through SIRPα because activation of SIRPα would inhibit phagocytosis. Instead, suitable soluble CD47 polypeptides facilitate the phagocytosis of endogenous HCSPs. A soluble CD47 polypeptide can be fused to an Fc (e.g., as described in US20100239579).

Immunotherapeutic reagents also include soluble SIRPα polypeptides specifically binding to CD47 and inhibiting its interaction with SIRPα. Exemplary agents include ALX148 (Kauder et al., Blood 2017 130:112) and TTI-622 and TTI-661 (Trillium). Such agents can include the entire SIRPα ECD or any portion thereof with the above functionality. The SIRPα reagent will usually comprise at least the d1 domain of SIRPα. The soluble SIRPα polypeptide can be fused to an Fc region. Exemplary SIRP a polypeptides termed “high affinity SIRPα reagent”, which includes SIRPα-derived polypeptides and analogs thereof (e.g., CV1-hIgG4, and CV1 monomer are described in WO2013/109752. High affinity SIRPα reagents are variants of the native SIRPα protein. The amino acid changes that provide for increased affinity are localized in the d1 domain, and thus high affinity SIRPα reagents comprise a d1 domain of human SIRPα, with at least one amino acid change relative to the wild-type sequence within the d1 domain. Such a high affinity SIRPα reagent optionally comprises additional amino acid sequences, for example antibody Fc sequences; portions of the wild-type human SIRPα protein other than the d1 domain, including without limitation residues 150 to 374 of the native protein or fragments thereof, usually fragments contiguous with the d1 domain; and the like. High affinity SIRPα reagents may be monomeric or multimeric, i.e. dimer, trimer, tetramer, and so forth. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more. Immunotherapeutic agents directed at CD47 or SIRPα with an Fc region can have any of the human isotypes, e.g., IgG1, IgG2, IgG3 or IgG4. Human IgG4 or IgG2 isotype or IgG1 mutated to reduce effector functions can be used because effector functions are not required for inhibiting the CD47-SIRPα interaction.

Immunotherapeutic agents, including antibodies and Fc fusion proteins, are administered in a regime effective to achieve the desired purpose of reducing or eliminating endogenous HSPCs. An effective regime refers to a combination of dose, frequency of administration and route of administration. The effective dose of such an agent can vary with the agent. Exemplary doses for an anti-c-Kit antibody are at least 0.05 mg/k and up to 10 mg/kg e.g., about 0.05-10 mg/kg, or 0.1 to 5 mg/kg. Exemplary doses for immunotherapy agents inhibiting CD47-SIRPα are at least any of 0.05 mg/kg, 0.1 mg/kg, 0.5 mg/kg, 1 mg/kg up to any of 5 mg/kg, 10 mg/kg, 20 mg/kg, 30 mg/kg 40 mg/kg or 50 mg/kg. Some exemplary ranges are 0.05 mg/kg-50 mg/kg, 0. 1 mg/kg to 20 mg/kg or 1 mg/kg to 10 mg/kg. Optionally, such immunotherapeutic agents can be administered initially at one or more priming doses, followed by one or more therapeutic doses to reduce undesired crosslinking of red blood cells, as described by e.g., WO02017181033.

The immunotherapy agent(s) can be administered one or more multiple time before introduction of replacement HSPCs to reduce endogenous HSPCs to a desired level or eliminate endogenous HSPCs. The regime can begin e.g., a month, two weeks, a week or less before introduction of replacement HSPCs. The immunotherapy agent(s) can also be administered one or multiple times after introduction of replacement HSPCs to select for HSPCs against residual endogenous HSPCs. Alternatively, the ablation regime can begin at the same time or after introduction of replacement HSPCs.

If multiple immunotherapeutic agents are used, the agent or combination of agents may or may not be the same before and after introduction of replacement HSPCs. For example, anti-c-Kit can be administered alone before introduction of replacement HSPCs and both anti-c-Kit and anti-CD47 afterwards. After introduction of replacement HSPCs, an ablation regime against endogenous HSPCs can be continued until endogenous HSPCs have been reduced to a desired level. Introduction of genetically modified HSPCs into a subject retaining some endogenous HSPCs with ongoing selection for the genetically modified HSPCs is advantageous in not completely depriving a subject of HSPCs with consequent risk of infection at any time.

Immunotherapeutic agents are typically administered as pharmaceutical compositions in which the agent is combined with one or more pharmaceutically acceptable carriers. A variety of aqueous carriers can be used, e.g., buffered saline and the like. These solutions are sterile and generally free of undesirable matter. These compositions may be sterilized by conventional techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and is selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

V. General Characteristics of Antibodies

The production of other non-human monoclonal antibodies, e.g., murine, guinea pig, primate, rabbit or rat, against an antigen can be accomplished by, for example, immunizing the animal with the antigen or a fragment thereof, or cells bearing the antigen. See Harlow & Lane, Antibodies, A Laboratory Manual (CSHP NY, 1988) (incorporated by reference for all purposes). Such an antigen can be obtained from a natural source, by peptide synthesis or by recombinant expression. Optionally, the antigen can be administered fused or otherwise complexed with a carrier protein. Optionally, the antigen can be administered with an adjuvant. Several types of adjuvant can be used as described below. Complete Freund's adjuvant followed by incomplete adjuvant is preferred for immunization of laboratory animals.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. Nos. 5,859,205 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5 CDRs from a mouse antibody) (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions.

A human antibody can be isolated from a human, or otherwise result from expression of human immunoglobulin genes (e.g., in a transgenic mouse, in vitro or by phage display). Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397, 5,874,299, 5,814,318, 5,789,650, 5,770,429, 5,661,016, 5,633,425, 5,625,126, 5,569,825, 5,545,806, Nature 148, 1547-1553 (1994), Nature Biotechnology 14, 826 (1996), Kucherlapati, WO 91/10741 (1991) and phage display methods (see, e.g. Dower et al., WO 91/17271 and McCafferty et al., WO 92/01047, U.S. Pat. Nos. 5,877,218, 5,871,907, 5,858,657, 5,837,242, 5,733,743 and 5,565,332.

Antibodies are screened for specific binding to their intended target. Antibodies may be further screened for binding to a specific region of the target (e.g., containing a desired epitope), competition with a reference antibody, agonism or antagonism of cells bearing the antigen. Non-human antibodies can be converted to chimeric, veneered or humanized forms as described above.

The choice of constant region depends, in part, whether antibody-dependent cell-mediated cytotoxicity, antibody dependent cellular phagocytosis and/or complement dependent cytotoxicity are desired. For example, human isotypes IgG1 and IgG3 have complement-dependent cytotoxicity and human isotypes IgG2 and IgG4 do not. Light chain constant regions can be lambda or kappa. Human IgG1 and IgG3 also induce stronger cell mediated effector functions than human IgG2 and IgG4.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes. Reference to a human constant region includes a constant region with any natural allotype or any permutation of residues occupying polymorphic positions in natural allotypes.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428, S or N at position 434, Y at position 252, T at position 254, and E at position 256. N434A (EU numbering). Increased FcRn binding is advantageous in making the hybrid proteins of the present invention compete more strongly with endogenous IgG for binding to FcRn. Also numerous mutations are known for reducing any of ADCC, ADP or CMC. (see, e.g., Winter et al., U.S. Pat. No. 5,624,821; Tso et al., U.S. Pat. No. 5,834,597; and Lazar et al., Proc. Natl. Acad. Sci. USA 103:4005, 2006). For example, substitution any of positions 234, 235, 236 and/or 237 reduce affinity for Fcγ receptors, particularly FcγRI receptor (see, e.g., U.S. Pat. No. 6,624,821). Optionally, positions 234, 236 and/or 237 in human IgG2 are substituted with alanine and position 235 with glutamine or glutamic acid. (See, e.g., U.S. Pat. No. 5,624,821.) Other substitutions reducing effector function include A at position 268, G or A at position 297, L at position 309, A at position 322, G at position 327, S at position 330, S at position 331, S at position 238, A at position 268, L at position 309. Some examples of mutations enhancing effector function include S239D, I332E, A330L and combinations thereof.

Antibodies of interest for ablation may be tested for their ability to induce ADCC. Antibody-associate ADCC activity can be monitored and quantified through detection of either the release of label or lactate dehydrogenase from the lysed cells, or detection of reduced target cell viability (e.g. annexin assay). Assays for apoptosis may be performed by terminal deoxynucleotidyl transferase-mediated digoxigenin-1 1-dUTP nick end labeling (TUNEL) assay (Lazebnik et al., Nature: 371, 346 (1994). Cytotoxicity may also be detected directly by detection kits, such as Cytotoxicity Detection Kit from Roche Applied Science (Indianapolis, Ind.). Antibodies can likewise be tested for their ability to induce antibody dependent phagocytosis (ADP) on for example AML LSC as described by WO/2009/091601.

In some embodiments, an immunotherapeutic agent is conjugated to an effector moiety. The effector moiety can be any number of molecules, including labeling moieties such as radioactive labels or fluorescent labels, or can be a cytotoxic moiety. Cytotoxic agents include cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin, saporin, auristatin-E and the like. Cytotoxic agents also include radiochemicals made by conjugating radioisotopes to antibodies. Targeting the cytotoxic moiety to transmembrane proteins serves to increase the local concentration of the cytotoxic moiety in the targeted area.

VI. Genetic Disorders of Blood Cells

The present methods can be used to correct genetic disorders of blood cells, particularly monogenic disorders arising from mutation of a single protein. Such disorders can be dominant or non-dominant and may result in partial or complete penetrance. In general such disorders can be treated by ablating endogenous HPLC and administering replacement HPLCs which include a functioning (e.g., wildtype) form of the protein underlying the disorder. Such cells can express the wildtype protein as well as or instead of the mutant form of the protein depending on how the genetic modification is carried out.

Genetic disorders of blood cells include hemoglobinopathies, such as thalassemias and sickle cell disease, X-linked severe combined immunodeficiency (X-SCID) adenosine deaminase deficiency (ADA-SCID), other genetic forms of SCID (artemis, Rag1/2), Wiskott Aldrich syndrome (WAS), chronic granulomatous disease, hemophagocytic lymphohistiocytosis, X-linked hyper IgM syndrome, X-linked lymphoproliferative disease, X-linked agammaglobulinemia, X-linked adrenoleukodystrophy, metachromatic leukodystrophy, hemophilia, von Willebrand disease, drepanocytic anemia, hereditary aplastic anemia, pure red cell aplasia, paroxysmal nocturnal hemoglobinuria, Fanconi anemia, hemophagocytic lymphohistiocytosis (HLH), inborn errors of metabolism, e.g., mucopolysaccharidosis, Gaucher disease and other lipidoses, epidermolysis bullosa, severe congenital neutropenia, Shwachman-Diamond syndrome, Diamond-Blackfan anemia, Kostmann's syndrome and leukocyte adhesion deficiency.

In sickle cells anemia, valine is substituted for glutamic acid in the sixth amino acid of the hemoglobin beta chain. The valine mutant form of hemoglobin is much less soluble than the glutamic form; it forms a semisolid gel of rod-like factoids that cause RBCs to sickle at sites of low P02. Distorted, inflexible RBCs adhere to vascular endothelium and plug small arterioles and capillaries, which leads to occlusion and infarction. Because sickled RBCs are too fragile to withstand the mechanical trauma of circulation, hemolysis occurs after they enter the circulation. In homozygotes, clinical manifestations are caused by anemia and vaso-occlusive events resulting in tissue ischemia and infarction. Growth and development are impaired, and susceptibility to infection increases. Anemia is usually severe but varies highly among patients. Sick cell anemia can be remedied by correcting the genetic defect, expressing an additional functional hemoglobin transcriptional unit or disruption of the BCL11A erythroid enhance, which represses fetal globin expression resulting in increased levels of fetal hemoglobin for treatment of sickle cell anemia (or beta thalassemia).

Thalassemias are a group of chronic, inherited, microcytic anemias characterized by defective hemoglobin synthesis and ineffective erythropoiesis, particularly common in persons of Mediterranean, African, and Southeast Asian ancestry. Thalassemia is among the most common inherited hemolytic disorders. It results from unbalanced Hb synthesis caused by decreased production of at least one globin polypeptide chain (β, α, γ, δ). This can occur through mutations in the regulatory regions of the genes or from a mutation in a globin coding sequence that results in reduced expression.

Combined immunodeficiency is a group of disorders characterized by congenital and usually hereditary deficiency of both B- and T-cell systems, lymphoid aplasia, and thymic dysplasia. The combined immunodeficiencies include severe combined immunodeficiency, Swiss agammaglobulinemia, combined immunodeficiency with adenosine deaminase or nucleoside phosphorylase deficiency, and combined immunodeficiency with immunoglobulins (Nezelof syndrome). Most patients have an early onset of infection with thrush, pneumonia, and diarrhea. If left untreated, most die before age 2. Most patients have profound deficiency of B cells and immunoglobulin. The following are characteristic: lymphopenia, low or absent T-cell levels, poor proliferative response to mitogens, cutaneous anergy, an absent thymic shadow, and diminished lymphoid tissue. Pneumocystis pneumonia and other opportunistic infections are common.

The present methods can also be used be used for treatment of infectious disease by modifying an immune cell receptor used by infecting viruses, such as CCR5 in the case of HIV.

These present methods can also be used to treat hematologic malignancies and autoimmune diseases in which the pathology at least in part resides in blood cells. Hematologic malignancies include leukemia, lymphomas and myelomas. More specific examples of such malignancies include multiple myeloma, Non-Hodgkin lymphoma, Hodgkin disease, acute myeloid leukemia, acute lymphoid leukemia, acute lymphoblastic leukemia, chronic myeloid leukemia; chronic lymphocytic leukemia, myeloproliferative disorders, and multiple myeloma. Autoimmune disorders include B and T-cell mediated disorders. Common examples are rheumatoid arthritis, systemic lupus erythematosus, inflammatory bowel disease, multiple sclerosis, type 1 diabetes, Guillain Barre syndrome, chronic inflammatory demyelinating polyneuropathy, psoriasis, Grave's disease, Hasimoto's thyroiditis, myasthenia gravis, vasculitis and systemic sclerosis.

The present methods can also be used for replacing endogenous HSPCs in patients with other types of cancer, such as solid tumors, who have received chemotherapy causing damage to endogenous HSPCs. Solid tumors include those of breast, prostate, brain, lung, kidney, liver, stomach, intestine, colon, thyroid, thymus, ovary, melanoma, and pancreas among others. Replacement stem cells supply the function of endogenous HSPCs (e.g., in fighting infection) and if allogenic may have additional activity against residual cancer cells.

The present methods can also be used for replacing HSPCs in organ transplants, particularly allografts. Endogenous HSPCs are likely to develop a host verses graft response against non-MHC-matched allografts. The host versus graft response can be reduced by ablating endogenous HSPCs before the organ transplant and introducing replacement HSPCs genetically modified to confer a proliferation advantage at the same time as the transplanted organ and preferably from the same source (i.e., subject).

Selection between autologous and allogenic sources for replacement HSPCs depends on several factors. Autologous transplantation is readily available, and there is no need to identify an HLA-matched donor. Autologous transplants have a lower risk of life-threatening complications; there is no risk of GVHD and no need for immunosuppressive therapy to prevent GVHD and graft rejection. Immune reconstitution is more rapid than after an allogeneic transplant and there is a lower risk of opportunistic infections. Graft failure occurs rarely. However, there is a risk that autologous transplants from cancer patients are contaminated with cancer cells.

Allogeneic transplantation has the advantage that the graft is free of contaminating tumor cells. The graft also includes donor-derived immunocompetent cells which may produce an immune graft-versus-malignancy effect. There is generally a lower risk for disease recurrence after allogeneic transplants compared to autologous transplantation. However, allogeneic transplants may be associated with a number of potentially fatal complications such as regimen-related organ toxicity, graft failure, and graft-versus-host disease.

In general, allogeneic transplants have been used predominantly in the treatment of leukemias and myelodysplastic syndromes. Autologous transplants have been used more often in solid tumors, lymphoma, and myeloma. For correction of genetic disorders, autologous transplants can be bused with genetic modification to correct the genetic basis for the disorder or allogenic transplant without the need for correction.

VII. Genetically Engineering of HSPCS

HSPCs are genetically modified to allow them to express one or more proteins providing a selective advantage against endogenous HSPCs in a subject. HSPCs can also be genetically modified to express a functional form of a protein that is deficient in endogenous HPLCs to treat a genetic disorder underlying the deficiency. Genetic modification can involve introduction of an exogenous nucleic acid encoding a receptor or other protein to be expressed. Such an exogenous nucleic acid can then exist as an episome or preferably be incorporated into the genome of genetically modified HSPCs. Such incorporation can be random or targeted, usually to a corresponding endogenous sequence. The exogenous nucleic acid can include regulatory sequences such as a promoter flanking the sequence to be expressed, or the sequence to be expressed can be designed to integrate at a chromosomal location in operable linkage with endogenous regulatory sequences. In either format, the genome of the HSPCs is modified to contain a transcriptional unit capable of expressing a receptor conferring a selective advantage or functional form of a protein deficient in endogenous HSPCs.

Genetic modification can also include introduction of a gene targeting construct to modify an endogenous gene encoding a receptor or protein, for example remove a mutation underlying a genetic deficiency, or inactivate an endogenous gene. Such modification is generally effected by recombination between the targeting construct and the endogenous allele. The targeting construct typically includes a nucleic acid to replace a segment of endogenous nucleic acid (e.g., with a wildtype codon in place of a mutant codon) flanked by homology arms to mediate homologous recombination. The frequency of targeted by modification can be increased by targeted cleavage proximate to the recombination effected by CRISPR, or a zinc finger protein, talon or the like fused to a nuclease domain (Shim et al., Acta Pharmacologica Sinica volume 38, pages 738-753 (2017)); U.S. Pat. Nos. 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983 and 20130177960.

Genetic modification can also involve activating or repressing expression of an endogenous receptor or protein. Expression can be activated or repressed by introducing a construct expressing a fusion protein of a DNA binding domain and a transcriptional activator or repressor (see e.g., U.S. Pat. No. 6,933,133). The DNA binding domain can be a zinc finger protein, talen or Cas9 mutated so as to bind to a target site without cleaving.

Genetic modification involves introduction one or multiple modifying elements into a cell (e.g., a targeting vector to effect modification and nuclease to promote homologous recombination, or a viral vector encoding a transcriptional unit to effect random insertion). If multiple elements are introduced, they can be included within the same or different construct. Likewise if multiple modifications are made (e.g., introducing both a nucleic acid encoding a protective receptor and a nucleic acid encoding a wildtype form of a protein to correct a genetic disorder), the modifying nucleic acids can be included in the same or different vectors and if the latter, the modifications can be performed at the same time or sequentially. HSPCs after genetic modification can be a clonal population or polyclonal. Genetic modification can result in a cell heterozygous or homozygous for the modification. Cells can undergo propagation before introduction into a subject.

Many vectors useful for transferring exogenous genes into target mammalian cells are available (see, e.g., WO2018/140940; Morgan et al., Cell Stem Cell 21, 574-590(2018)). The vectors may be episomal, or may be integrated into the target cell genome, through homologous recombination or random integration. Vectors may or may not encode a selectable marker to select for modified cells. Vectors include plasmids, virus derived vectors such cytomegalovirus, adenovirus and so forth, retrovirus derived vectors such MMLV, HIV-1, and ALV. Lentiviral vectors such as those based on HIV or FIV gag sequences can be used to transfect non-dividing cells, such as the resting phase of HSPCs.

Cells may be genetically altering by transfection (e.g., electroporation), transduction, or the like with a suitable vector. Combinations of retroviruses and an appropriate packaging line can be used where the capsid proteins are functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line or transfection, cells may be genetically altered, for example, using vector containing supernatants over an 8-16 h period, and then exchanged into growth medium for 1-2 days, optionally with selection using a drug selection agent such as puromycin, G418, or blasticidin, and then recultured. Viral or plasmid vectors may include genes that must later be removed, e.g. using a recombinase system such as Cre/Lox, or the cells that express them destroyed, e.g. by including genes that allow selective toxicity such as herpesvirus TK, bcl-xs, among others.

VIII. Regimes for Administering Replacement Stem Cells

Replacement stem cells are administered parenterally, typically by intravenous infusion. The dose of stem cells administered can depend on the desired purity of the infused cell composition, and the source of the cells. The dose can also depend on the type of genetic modification of the HSPCs. Because of the protection of HSPCs and because substantially complete elimination of endogenous HSPCs before introduction of replacement HSPCs is not necessary, dosages can sometimes be less than in prior methods in which 1-2×10⁶ CD34+ cells/kg body weight was considered a minimum. Exemplary dosages of cells for reintroduction are at least 1×10⁵, 1×10⁶, 2×10⁶, 5×10⁶, 10⁷, 2×10⁷ CD34+ cells/kg body weight. Exemplary range are 1×10⁵ to 5×10⁷, 1×10⁶ to 2×10⁷, or 5×10⁵−6×10⁶ CD34+ cells/kg body weight. The dose may be limited by the number of available cells. Typically, regardless of the source, the dose is calculated by the number of CD34+ cells present. The percent number of CD34+ cells can be low for unfractionated bone marrow or mobilized peripheral blood; in which case the total number of cells administered is much higher.

VIII. Monitoring

After introduction of genetically modified replacement HSPCs into a subject, the ratio of replacement HSPCs to total HSPCs can be monitored. A sample of HSPCs can be obtained from bone marrow or peripheral blood as previously described. Replacement HSPCs can be distinguished from endogenous by e.g., a nucleic acid hybridization assay or immunoassays. If the replacement HSPCs are allogenic or xenogenic, there are many genetic differences a between the replacement and endogenous cells that can form the basis of a differential probe binding assay and sometimes differences in receptors that allow an immunoassay. If the replacement HSPCs are autologous, the genetic modification of the replacement HSPCs can distinguish them from endogenous HSPCs by either a nucleic acid hybridization assay or immunoassay. The proportion of replacement to total HSPCs may increase with time after introduction. Preferably the proportion exceeds 30, 50, 75, 90 or 95% after six months.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

What is claimed is:

1. A hematopoietic stem or progenitor cell (HSPC) genetically modified to express a receptor conferring a selective proliferation advantage on the genetically modified HSPC on introduction into a subject relative to endogenous HSPCs.

2. A population of at least 10⁵ HSPCs of claim 1.

3. The population of claim 2, wherein the 10⁵ HSPCs are CD34⁺.

4. The population of claim 2 that is clonal.

5. The population of claim 2 including primitive stem cells and common progenitor cells.

6. The HSPC of claim 1 that is a primitive stem cell.

7. The HSPC of claim 1, wherein the receptor is any of B2M/MIHC-1, PD-L1, CD24, GAS6, CD47, c-Kit or a combination of multiple such receptors.

8. The HSPC of claim 7, wherein the receptor is a mutant form, wherein the mutant has reduced binding to an antibody relative to the wildtype form of the receptor.

9. The HSPC of claim 8, wherein the receptor is CD47 or c-Kit.

10. The HSPC or population of claim 1, wherein the HSPC is further genetically modified to express a functional human protein as a result of which the HSPC can alleviate a genetic disorder.

11. The HSPC or population of claim 10, wherein the genetic disorder is due to mutation of a gene encoding the human protein in subjects having the disorder.

12. The HSPC or population of claim 11, wherein the human protein is a hemoglobin.

13. The HSPC or population of claim 1, wherein the HSPC is genetically modified by homologous recombination between a targeting construct and an endogenous locus.

14. The HSPC or population of claim 1, wherein the genetic modification is heterozygous.

15. The HSPC or population of claim 1, wherein the genetic modification is homozygous.

16. A method of modifying an HSPC comprising introducing into the HSPC a construct that is incorporated into the genome of the HSPC forming a transcriptional unit that can express a receptor conferring a selective proliferation advantage on the genetically modified HSPC relative to the HSPC before modification.

17. The method of claim 16, wherein the construct comprises a transcriptional unit comprising a segment encoding the receptor operably linked to regulatory sequences for its expression.

18. The method of claim 16, wherein the construct undergoes homologous recombination with an endogenous locus.

19. The method of claim 16, further comprising introducing a nuclease into the HSPC, which cleaves genomic DNA proximate to the locus of the homologous recombination thereby stimulating the homologous recombination.

20. The method of claim 19, wherein the nuclease is introduced by introducing a construct encoding the nuclease, which is expressed in the HSPC.

21. A method of treating a subject, comprising
(a) administering an immunotherapeutic agent specifically binding to c-Kit to deplete endogenous HSPCs expressing c-Kit; and
(b) administering replacement HSPCs genetically modified to express a receptor conferring a selective proliferation advantage on the genetically modified HSPCs relative to endogenous HSPCs and thereby resist depletion by the immunotherapeutic agent specifically binding to c-Kit, wherein the replacement HSPCs at least partially replace the endogenous HSPCs.

22. The method of claim 21, wherein the receptor conferring a selective proliferation advantage is CD47.

23. The method of claim 22, wherein the CD47 receptor contains a mutation and the method further comprises administering an antibody or SIRPα Fc fusion protein that binds to wildtype CD47 and antagonizes its interaction with SIRPα more strongly over its binding and antagonism, if any, of the mutated receptor to SIRPα.

24. The method of claim 21, wherein endogenous HSPCs are only partially depleted before performing step (b).

25. The method of claim 21, wherein step (a) is performed before step (b).

26. The method of claim 21, wherein step (a) is performed at the same time or after step (b).

27. The method of claim 21, wherein the immunotherapeutic agent specifically binding to c-Kit is detectable in the serum when the introducing step is performed.

28. The method of claim 21, wherein the immunotherapeutic agent specifically binding to c-Kit is administered on multiple occasions before and after step (b).

29. The method of claim 21, wherein the immunotherapeutic agent specifically binding to c-Kit is an antibody.

30. The method of claim 21, wherein the antibody has an Fc domain effective to promote ADCC or ADP.

31. The method of claim 21, wherein the subject has a genetic disorder of a type of blood cell and the replacement HSPCs develop into blood cells of the type free of the disorder.

32. The method of claim 21, wherein the replacement HSPCs are autologous cells which have been further genetically modified to be free of the disorder.

33. The method of claim 31, wherein the genetic disorder is sickle cell anemia.

34. The method of claim 21, wherein the subject has a cancer.

35. The method of claim 34, wherein the cancer is of a blood cell, which expresses c-Kit or derives from a HSPC expressing c-Kit.

36. The method of claim 21, wherein the subject has a cancer and has received chemotherapy against the cancer.

37. The method of claim 21, wherein the subject receives an organ transplant after step (a).
